(19) United States
(12) Patent Application Publication
(10) Pub. No.: US 2020/0042917 A1
(43) Pub. Date: Feb. 6, 2020

(54) Solution Oriented Conglomerate building implementing sub organizations offering services and products with strategies, solutions and products in multiple industries (health are, education, business, government, housing , entertainment)

(71) Applicant: Natalie Rodrigues, Forest Hills, NY (US)

(72) Inventor: Natalie Rodrigues, Forest Hills, NY (US)

(21) Appl. No.: 16/053,237

(22) Filed: Aug. 2, 2018

(51) Int. Cl. G06Q 10/06; A21D 13/41; G06Q 40/00; G06Q 50/16; G06Q 50/18; G06Q 50/20; G16H 10/60; G16H 40/20

(52) U.S. Cl. CPC G06Q 10/06313; A21D 13/41; G06Q 40/123; G16H 40/20; G06Q 50/18; G06Q 50/205; G16H 10/60; G06Q 50/165

(57) ABSTRACT

Fatima Partners, LLC is a solution oriented management company overseeing organizations that provide alternatives, reforms, or solutions in various industries, including but not limited to education, health care, entertainment, publishing, and business, etc.

The solutions are presented in the form of products, services, policies, and strategies that aim to make organizations, professionals and products more relevant and meaningful to the client. Aiming to meet the actual not the partial needs of the consumer, Fatima Partners, LLC has developed unique business methods, products, policies and solutions.

Starting on a modest scale, with the potential to add partners, new organizations, products and services in the future Fatima Partners, LLC is has and will continue to offer micro and macro level solutions that can be create and implemented overnight or overtime. It also has the potential to expand and grow to impact multiple industries, aspiring inventors, reformers and consumers across the nationally and across the globe.

The inventor is seeking to protect the intellectual property to minimize interference and potential misinformed or obstructive influence within the development and promotion of policies, products and services to relevant markets.

The solutions, and the solution oriented strategies, methods, processes, systems, are patentable (as some products and services listed in this patent have previously been patented by other inventors using different ingredients, formulas, processes, and methods/ie: pizza, medical reporting etc).

### Chart A

1. Project Plan Your Year

2. Project Plan for Teachers: Organize Your Classroom for Success

3. Project Plan Your Time & Money

4. The Enlightened Evaluation: A Mental Health Evaluation Framework for Responsible Reporting

5. Career Development for Teachers: What's Your Next Best Move

6. Goals & Gratitude: Goal Setting for the New Year

Planned and proposed books to be published over the next several years are listed on our website.
Chart B: See workshop lists below

1. Project Plan Your Year

2. Project Plan for Teachers: Organize Your Classroom for Success

3. Project Plan Your Time & Money

4. The Enlightened Evaluation: A Mental Health Evaluation Framework for Responsible Reporting

5. Career Development for Teachers: What's Your Next Best Move

6. Goals & Gratitude: Goal Setting for the New Year Goal Setting for the New Year

With more coming
Trial workshops attached.

1. Our business method incorporates multiple ‘projects’which, finds new uses for pre-existing spaces; creates and implements innovative business methods and product models; designs improved processes on current products and services, or develops alternative methods to inaccurate, unethical, inconsistent, or ineffective frameworks, policies, products or services; offers an effective combination of product or organizational components.

2. In combination with claim 1, our business method consists of, incorporating cross learning, economies of scale through shared strategies, space, costs and resources, amongst and between two similar and/or dissimilar projects, products or services.

3. In conjunction with claim 1 and 2, we implement business goals by offering several consultant companies to choose from based on focus, objectives and audience (see Appendix A); our process includes, providing upper level, mid management, and team level consultant services, including ‘temp-like’ support services, which focuses on customer service, human capital development, employee satisfaction, and building or reforming for ‘relevant systems’; investing in human capital through a commitment to incorporating enriching professional development and trainings (see claim 9), creating forums for collaboration and building partnerships amongst ‘projects’ or departments (when consulting within Fatima Partners and outside), addressing inefficiencies and problem areas by addressing root causes and creating informed plans from either original solutions, industry based research or external industry strategies; packaging unlikely and random strategies for appeal and effective results, and renaming committed to human capital as important inventory ensuring for effective HR strategies to retain employees.

4. In combination with claims 1, 2 and 3, the consulting business method (provisional patent 62/761,240) incorporates an unique experience and marketing methods which advertise pre-designed services for the predicted asks of potential clients with “memorable and rhythmic names” that are copyrighted (2018), as well as slogans to allow for customization of services during moments when the company is in preventative and planning stages rather than reactionary modes.

5. In combination with claim 1, 2, 3, 4 the consulting business method also includes the development of a publishing arm to offer pre-designed copyrighted training materials, personal and professional development workbooks or frameworks (see claim 9 and provisional patent 62/761,239) which release information on new industry concepts, business methods, systemic solutions and/or alternatives, and proposed policies. (several products copyright and/or published are listed in Chart A.

6. In conjunction to claim 1, 2, 3, 4, 5 pre-designed frameworks listed in referenced in patent 62/643,150 and have been published in book format include, the Enlightened Evaluation: Mental Health Evaluation Process for Responsible Medical Reporting (MHEP), lists the micro process being patented in this application as an more effective method of evaluating patients to avoid irresponsible medical reporting and misdiagnosis, by providing a process for organizing and recording ‘findings’, effectively diagnosing and selecting appropriate treatment for patients by focusing on root causes, observing behaviors, thoughts and emotions, considering both medical and mental health symptoms (to identified co-existing health concerns) when diagnosing patients, ‘measuring maslow’ otherwise known as conducting a needs assessment, understanding patients history and ‘ways of being’, goal setting to engage, provide ownership in one's health and hold patients accountable during their evaluation, treatment and overall health improvement.

7. In combination with the claim above 6, the process for diagnosing and selecting more accurate and targeted treatment plans include, working with the patient to create an initial observation or thesis on the patient; identify a patient's life history, philosophies and ways of being, behaviors, behaviors in treatment, emotions, statements, thoughts in order to obtain a broad picture of the patient internally and externally; identify patient supports, physical needs, social emotional needs, aspirational needs, symptoms—medical, symptoms—mental, mental systems caused by medical for more accurate diagnosing; conduct constructive/destructive analysis of the patients moo and personality; create accountability by outlining treatment goals, medicine selection, treatment plan (MHP tasks), patient tasks (ownership), prognosis.

8. In conjunction to claim 1, 2, 4, 5, 6, 7 our organization seeks to protect a solution based alternative to contemporary talk therapy in the mental health field, that consists of incorporating dual methods (business and healthcare) consisting of movement or activity to promote healthy coping mechanisms, symptom or emotional alleviation, modify moods, restore joy, and to infuse a process centered around problem solving of past and current issues in order to move forward with planning and meeting future goals (see also Project Plan Series, as an additional aid claim 9).

9. In conjunction to claim 1, 2, 3, 4, 5, 6, 8, the Project Plan Series is a process, teaching method, and self-organizational framework located within books and workshops that has been provisional patented 62/761,239 and published as a workbook and workshops in 2019/2018 (Chart A and B), and as a product to aid to previously mentioned consulting services, the ‘School of Curiosity’, ‘Activity Therapy, the Empowerment Center and a complementary or standalone product to support proposed educational and healthcare solutions mentioned in this patent. Product components include informative description, analogies, questioning, reflection, cases, charts, organizers, tables, calendars, task lists, project plans, to organize and goal set as a process to prepare and implement for change.

10. In conjunction to claim 1, 2, 5, 7, 8, 9 the School of Curiosity is a school design that includes a school model method which includes a combination of strategies, curriculum, services, and focus points called 10 tenants which include, (incorporating Curiosity in the curriculum and school structure, as well as PPS motivational workbook-curricula (claim 7), Special Services (including claim 6), Parent Engagement, Teacher Development, Gaming, Global Citizenship, Strategic Partnerships, Citizen-Consumer Classes, Behavior Modification/Rigorous Physical Education, Creative Literacy, and Technology, built into a school structure (schedule, building, curriculum, mission, teaching practice, etc) to support student academic success.
Description of tenants in details listed within the patent.

11. In conjunction to claim 1, 2, 3, 5, 10 a process entitled ‘School Leader Goal List’ is a solution based framework and product published as an alternative to evaluation systems promoting a method of reflective and an evidence based accountability tool to measure the development and acquisition of skills and attributes identified as being present in an effective school leader. The process includes reflecting on identified attributes, quantifying and qualifying school leader effectiveness with observation, data and artifacts, detailing the impact on the school, students, staff etc., and developing a plan of action to refine skills.

12. In conjunction to claims 1, 2, 3, 5 other consulting created documents including the Massaro Supreme Court Summons & Complaint Method which consists of describing expectations of a response, overall complaint, legal issues, laws breaches, relevant precedence's, damages, restitution/restoration and conclusion.

13. In conjunction to claims 1, 2, 3, 4, 5, 9 a consultant created solution to economic reform including incorporating new methods of generating revenue for a district, city, state, country that includes, promoting the production of more goods by government then services by nationalizing already highly demanded products, increase the privatization of human services to alleviate both the financial burden of funding endeavors and addressing areas where the government does not hold a competitive advantage, providing incentive to save and eliminate wasteful spending by allowing for the recycling of funds from previous years.

14. In conjunction to claim 1, 2, 3, 4, providing a solution to housing shortages, home ownership costs, and a mortgage crisis in communities that often are ‘targeted’ in unethical financial schemes consisting of building “tiny houses” (200 sq. ft-1200 sq. ft) in a rent to own process in order to replace the risk and pressure of taking on the commitment of a mortgage individuals may opt to pay rent until an agreed home prices is paid off in installments, if completion of all payments are fulfilled individuals will be awarded the deed and the responsibility of owning the home, individuals are also able to expand their home block by block or room by room.

15. In conjunction to claim 1, 2, 3, 14 Rodrigues Housing Design, is a method of building a smaller size home that is expandable by designing the home so that rooms can be added ‘block by block’, instead of brick by brick

16. In conjunction with claim 1, 2, 3, 4, 5, 6, 7, 8, 9 and developed around a method of business which utilizes pre-existing space in new ways and with multiple methods of generating revenue; for instance, the Empowerment Center provisional patent 62/761,238 seeks to combine complimentary business services (like Walmart) into a one stop shop for fitness, health, self/career development, women/community empowerment, and spiritual offerings into one center creating a multi-tier fee structure. The method being offering several identified services, not just membership to raise revenue.

17. Combined with claim 1, 14, 16, 18, 20 Spartan Theatre is a pre-existing closed down theatre that when implementing a new business model would include a combination of product/service offerings including hosting old and new movies, computer gaming, rental of stage/theatre for alternative entertainment (live-performances) and community uses, food offerings, traditional and nontraditional snacks such as, Mayan Pizza (See claim 18); in summary one stop shop for multiple streams of entertainment in the community

18. Combined and in conjunction with claim 1, 2, 14, 18 . . . a music venue dedicated to providing live music by street musicians, raw talent, and popular artists utilizing a business method of shared profits—giving the musicians themselves a stake in business development and growth.

19. In conjunction with claim 1, 2, 10, 14, 16, 17, 18 which seeks to implement a business method of identifying novel uses of pre-existing inventions or totally new methods of creating traditional items such as, ‘Mayan Pizza’ a pizza consisting of tortilla, topped with pizza sauce and mozzarella cheese, baked on a baking sheet with oil at 350 degrees Fahrenheit for 8-12 minutes. (Served at Spartan Theatre, Phoenix Music Venue, Empowerment Center)

20. In conjunction with claim 1, 2, 3, 4, 14, 15, 16, 17, 18, 19, a computing center for mobile professional includes a business method offering office services, computer rentals, printing, small conference room space, short term office rental space, and light catering.
